(19) United States
(12) Patent Application Publication
(10) Pub. No.: US 2005/0142187 A1
(43) Pub. Date: Jun. 30, 2005

(54) Enhanced absorption of modified release dosage forms

(76) Inventors: Donald J. Treacy JR., Annapolis, MD (US); Henry H. Flanner, Montgomery Village, MD (US); Robert J. Guttendorf, Gaithersburg, MD (US); Beth A. Burnside, Bethesda, MD (US)

Correspondence Address: Raymond E. Stauffer, Esq.;c/o Carella, Byrne, Bain, Gilfillan,, Cecchi, Stewart & Olstein, 5 Becker Farm Road, Roseland, NJ, 07068, US

(21) Appl. No.: 11/021,309

(22) Filed: Dec. 23, 2004

Related U.S. Application Data
(60) Provisional application No. 60/532,772, filed on Dec. 24, 2003.

(51) Int. Cl. A61K 31/43; A61K 9/48; A61K 9/20

(52) U.S. Cl. 424/451; 424/464; 514/192

(57) ABSTRACT

Disclosed are products and methods for improving the plasma profile in a patient being treated with a pharmaceutical active agent that is subject to a limited window of absorption, which products and methods comprise orally administering the active agent in multiparticulate form, such that at least a portion thereof is delivered to the intestine while the patient is in the fed condition.

This application claims the priority of U.S. Provisional Application Ser. No. 60/532,772 filed on Dec. 24, 2003, the disclosures of which are hereby incorporated by reference in their entireties.

This invention relates to a pharmaceutical product that includes a pharmaceutically active agent that has a limited window of absorption and to a method of treating patients with such a pharmaceutical product.

### BACKGROUND OF THE INVENTION

There is continuing need in the art, and continuing demand in the market, for pharmaceutical products that include, as an active ingredient, a drug with a limited window of absorption, more particularly there is a need for pharmaceutical products that can deliver such an active ingredient in a once-a-day dosage form. The need and demand notwithstanding, formulating once-a-day products containing narrow or limited window of absorption actives (actives which by pharmacokinetic necessity are generally administered more than once and up to several times in a day) continues to present significant challenges for the pharmaceutical formulator. Critically important to the safety and effectiveness of any pharmaceutical formulation is its ability to maintain a target blood level of the active pharmaceutical agent within active's the therapeutic concentration range. The therapeutic range, as known in the art, is bounded at the low end by the minimum concentration of active pharmaceutical agent necessary to elicit a therapeutic effect, and at the upper end by the blood concentration at which toxic side effects become limiting. Absorption barriers along the GI tract that slow or inhibit the absorption of certain active agents further limits the formulator, preventing the effective formulation of such absorption inhibited active agents into the more desirable once-a-day products. Additional pharmacokinetic parameters that must be evaluated when developing a once-a-day product include, but are not limited to area under the curve (AUC); Cmax, which is the highest blood level achieved; Tₘₐₓ, which is known as the time at which the blood level reaches the Cmax; and several additional parameters that may be useful to characterize the in vivo performance of a modified release formulation. Such additional parameters are partial area under the curve, mean absorption time, mean residence time, absorption constant, elimination constant, and other methods or metrics known to one skilled in the art.

A significant drawback when dosing drugs having a limited window of absorption through conventional modified release once-a-day dosage forms is the tendency of the drug to exhibit reduced area under the curve (AUC), compared to formulations of the same limited window of absorption drug dosed multiple times in a day. In general, for many active agents a loss in area under the curve can be observed in the pharmacokinetic profiles of such traditional modified release dosage forms. Frequently, such loss in area under the curve will prompt the formulator either to abandon hopes of developing a controlled release product or to reformulate the product with an increase in the dose of the immediate release portion, or to increase the overall daily dose required, but only after thoroughly studying the possibility of increased toxicity from increased exposure to the active agent. However, the increase in the immediate release dose often results in a higher Cmax, thereby increasing the likelihood of undesired side effects. Additionally, when the limited window of absorption drug has the further aspect of a short elimination half life, it is particularly difficult to maintain adequate therapeutic blood levels using modified-release, once-a-day regimens even when higher dosages are administered. The inadequate therapeutic blood level is due to the rapid clearance of the active pharmaceutical agent in combination with the limited ability that the drug agent has to be absorbed after a certain point in the GI tract.

The limited window of absorption presents a serious challenge to the development of effective modified-release preparations of these compounds. Because the length of time during which delivery may result in effective absorption is restricted to a limited window, a modified release dosage form's continued delivery of such drug beyond that limited window is rendered a nullity and results in a loss of bioavailability.

The widely-known poor or decreased absorption of these drugs may be attributed to a variety of barriers. The barriers presenting a small or limited window of absorption can be either biological or physico-chemical in nature, and can be, but are not limited to poor solubility, low permeability, and saturable active absorption or influx mechanisms such as carrier mediated transport.

Poor solubility over a broad pH range is another well known barrier that inhibits absorption and overall bioavailability for a number of compounds. Furthermore, when solubility is limited at the higher pH's found in the distal GI tract, a limited window of absorption is effectively created.

A significant consequence of a limited window of absorption is the inability to achieve Tₘₐₓ at a time beyond the outer limit of that window of absorption, typically 3-4 hours or less. Tₘₐₓ is the time at which the rate of absorption of an active agent into the bloodstream is equal to its rate of elimination from the bloodstream; it marks the moment in time that Cₘₐₓ is reached. Therefore, if an active drug agent's absorption window is limited to a certain time, typically 3-4 hours, Tₘₐₓ cannot be achieved beyond that time because absorption is not possible or is greatly reduced outside of that observed window of absorption. These limited windows of absorption significantly curtail the bioavailability and extent to which Tₘₐₓ can be extended using conventional modified release dosage forms known in the art.

### SUMMARY OF THE INVENTION

This invention relates to an improved pharmaceutical product, and method of use thereof, for delivery of a pharmaceutically active agent that has a limited window of absorption, wherein at least a portion of such product includes a modified release dosage form. In accordance with a further aspect of the invention the improvements are achieved by treating a patient with the pharmaceutical product while the patient is in the fed condition. In accordance with a still further aspect of the invention the product includes instructions that the product is to be administered to, or is to be taken by, the patient while the patient is in the fed condition. In accordance with a yet still further aspect of the invention the modified release dosage form is a dosage form that is formulated in a manner such that the dosage form is released from the stomach into the intestine as particulates, and such that at least a portion of the pharmaceutically active agent is released from the particulates into the small intestine.

The modified release dosage form aspect of the invention may be a delayed release dosage form, whereby the release of active agent therefrom is initially delayed until the dosage form reaches the small intestine at which time there is an immediate release of active agent. Alternatively or concomitantly, the modified release dosage form aspect of the invention may be a sustained release dosage form, whereby the release of active agent therefrom is initially delayed until the dosage form reaches the small intestine at which time there is release of active agent over an extended period of time. In one embodiment of the invention the pharmaceutical product comprises a combination of dosage forms at least one of which is the modified release dosage form as described hereinabove and hereinbelow. Additional dosage forms may include immediate release dosage forms, delayed release dosage forms, and/or sustained (extended) release dosage forms. These additional dosage forms may or may not include an active agent with a limited window of absorption as that term is known in the art.

The particulates comprising the modified release dosage form may be formed into a unitary pharmaceutical product, for example, in a capsule, or embedded in a tablet, or suspended in a liquid for oral administration. In addition to containing the particulates comprising the modified release dosage form such unitary pharmaceutical product may also contain additional dosage forms in those embodiments of the invention that contain a combination of dosage forms as hereinabove described. Alternatively, each of the dosage forms of the product may be formulated as a tablet, with each of the tablets being put into a capsule to produce a unitary pharmaceutical product. Alternatively the particulates comprising the modified release dosage form may be in the form of sprinkles to be added to food, such as applesauce. In the case where the pharmaceutical product is a tablet or capsule, it is designed and formulated to break down in the stomach. The pharmaceutical product includes a therapeutically effective amount of the pharmaceutically active agent, all or a portion of which may be in the modified release dosage form. The therapeutically effective amount will vary with the pharmaceutically active agent to be used, the disease or infection to be treated, and the number of times that the composition is to be delivered in a day. In one embodiment the pharmaceutical product is administered to a host in an amount effective for treating a bacterial infection.

As used herein, and as is generally known in the art, a pharmaceutically active agent described as having a “limited window of absorption” means that the pharmaceutically active agent is one that is essentially only absorbed in the small intestine. More particularly, as is further known in the art, most dosage forms, whether as tablets, pellets, capsules, particulates, or solutions, take about 3 to 4 hours to traverse the small intestine (G. Chawla et al., “Gastroretention A Means to Address Regional Variability in Intestinal Drug Absorption,” Pharmaceutical Technology, July 2003) whereby a pharmaceutically active agent having a limited window of absorption may be absorbed only during a period of 3 to 4 hours or less following release of the pharmaceutically active agent from the stomach.

### BRIEF DESCRIPTION OF THE DRAWING

FIG. 1 is a graph of mean plasma profiles.

### DETAILED DESCRIPTION OF THE INVENTION

The instant invention is directed to a pharmaceutical product, and method of use therewith, for treating a patient with a pharmaceutically active agent that has a limited window of absorption. The pharmaceutical product comprises an oral dosage unit (or a plurality thereof) comprising a modified release dosage form with or without an immediate-release portion, and instructions informing the user that the oral dosage unit is to be administered to, or is to be taken by, the patient while the patient is in the fed condition. The modified release portion of the dosage form is both designed and intended to be released from the stomach into the intestine as particulates. Those particulates release at least a portion of the pharmaceutically active agent, having the limited window of absorption, in the small intestine.

The pharmaceutical products and methods of the invention for treating a patient with a modified release formulation of a pharmaceutically active agent having a limited window of absorption, in one aspect, increase Tₘₐₓ for the limited window of absorption drug and such increase in Tₘₐₓ in accordance with an aspect of the invention is achieved without a decrease in bioavailability. The later achievement of Tₘₐₓ means that the point in time that the blood level of the active agent reaches the Cmax is achieved at a time that is later than the time that is the outer limit of the active agent's limited window of absorption.

More specifically, this invention allows for an improved plasma profile of a modified release formulation of a pharmaceutically active agent in a treated patient by administering to a patient, in the fed condition, a pharmaceutically active agent that is predominantly absorbed in a limited area of the GI tract, preferably the upper GI tract or the small intestine (i.e., a limited window of absorption agent), when the pharmaceutically active agent is in a modified release dosage form that is released from the stomach in multiparticulate form, and wherein at least a portion of the pharmaceutically active agent is released in the intestine.

In one aspect, the invention as thus described allows Tₘₐₓ for a limited window of absorption drug to be achieved at a later point in time than in the fasted state without significant loss of bioavailability. The delayed achievement of Tₘₐₓ provided by the invention results in the particular pharmaceutical active agent achieving and maintaining a minimum therapeutic concentration for a longer period of time than would otherwise be possible. Since the efficacy of many therapies are dependent upon the concentration of the active agent being maintained at or above a minimum therapeutic concentration (MTC) for a certain period of time, or alternatively, maintaining a concentration within the therapeutic range, the instant invention's prolonging of the time during which the active agent concentration is at or above MTC requires less frequent dosing to achieve and maintain efficacy. In accordance with an aspect of the invention, Tₘₐₓ can be extended and bioavailability can be maintained without increasing Cₘₐₓ, thus providing an improved safety and side effect profile.

In accordance with one aspect of the invention, there is provided an orally administered pharmaceutical product comprising a modified release dosage form that includes a pharmaceutically active agent and a pharmaceutically acceptable carrier combined, in multiparticulate form, wherein the active agent is a substrate for an active transporter, or wherein the active agent otherwise exhibits a limited window of absorption but is not a substrate for an active transporter. The modified release dosage form is formulated so that at least a portion of the multiparticulate combination of the pharmaceutically active agent and the pharmaceutically acceptable carrier are delivered to the intestine. It is a further aspect of the invention that the pharmaceutical product is administered to the patient while the patient is in the fed condition.

In accordance with the invention the pharmaceutical active agent is delivered to the intestine in multiparticulate form. In one embodiment the multiparticulate form of the pharmaceutical active agent is orally administered as a capsule dosage form. In one embodiment the multiparticulate form of the pharmaceutical active agent is orally administered as a rapidly disintegrating tablet or capsule dosage form that is immediate or delayed in its disintegration upon ingestion. In a further embodiment the multiparticulate form of the pharmaceutical active agent is administered by sprinkling on food, such as on applesauce, or into a liquid and ingested as a suspension.

In a preferred embodiment the active agent is in microparticulate form.

In general, administering the pharmaceutical product of the instant invention to a patient in the fed condition shall mean making such administration as currently understood in pharmaceutical, clinical, or medical practice. In another embodiment the pharmaceutical product of the instant invention shall be administered to the patient within the time period that is one half hour before and up to two hours after the patient has had a meal. In yet another embodiment the pharmaceutical product of the instant invention shall be administered to the patient with a high-fat, high-calorie meal such as described in Guidance for Industry: Food-Effect Bioavailability and Fed Bioequivalence Studies; U.S. Department of Health and Human Services, Food and Drug Administration, Center for Drug Evaluation and Research (CDER) December 2002, which is hereby incorporated by reference.

In one embodiment the pharmaceutically active agent has a limited window of absorption, typically no more than 6-8 hours. In a preferred embodiment the pharmaceutically active agent has a window of absorption of 3-4 hours or less. In a more preferred embodiment the pharmaceutically active agent is a substrate of an active transporter or is absorbed by a carrier mediated process. In a particularly preferred embodiment the active agent is a substrate of PEPT1.

In one embodiment the pharmaceutically active agent is an anti-infective. In a preferred embodiment the pharmaceutically active agent is an antibiotic. In a more preferred embodiment the pharmaceutically active agent is a beta-lactam antibiotic. In a particularly preferred embodiment the pharmaceutically active agent is amoxicillin and/or a pharmaceutically acceptable salt thereof. In another particularly preferred embodiment the pharmaceutically active agent is cephalexin and/or a pharmaceutically acceptable salt thereof.

In one embodiment the instant invention is practiced in accordance with PULSYS™, an oral drug delivery technology that enables once daily pulsatile dosing, as that technology is disclosed and embodied in U.S. Pat. No. 6,544,555 B2, the disclosures of which are hereby incorporated by reference in their entireties. In another embodiment the invention is practiced as a delayed release formulation. In another embodiment the invention is practiced as a sustained release formulation.

In one embodiment the pharmaceutical product contains more than one pharmaceutically active agent and or adjuvant. In one embodiment at least one of the pharmaceutically active agents is an inhibitor. In one embodiment at least one of the pharmaceutically active agents is an antibiotic. In a preferred embodiment at least one of the pharmaceutically active agents is a beta-lactam antibiotic. In another preferred embodiment wherein an inhibitor is present, the inhibitor is a beta lactamase inhibitor. In a more preferred embodiment at least one of the pharmaceutically active agents is amoxicillin and/or a pharmaceutically acceptable salt thereof.

The instant invention overcomes the prior art shortcoming of diminished bioavailability by extending the Tₘₐₓ while maintaining a similar area under the curve (similar to the that of a similar regimen dosed in the fasted state), thereby providing patients with greater time during which the active pharmaceutical agent is maintained within the necessary therapeutic range. Conventional once-a-day formulations tend to lose bioavailability and cannot extend Tmax sufficiently. The instant invention sufficiently extends Tmax, without a loss of bioavailability, and in some instances with an increase in bioavailability.

Applicants have found that the bioavailability and efficacy of certain antibiotics and therapeutic agents, agents known to have decreased absorption due to any or several of the above-described biological and physicochemical barriers, can be enhanced when such therapeutic agents are administered to a patient by way of a multiparticulate, modified-release dosage form, while the patient is in the fed state. Similarly, compounds that are converted to their active form via intestinal or hepatic first-pass metabolism such as some prodrugs, would also benefit by the invention.

Unexpectedly, it has been found that compounds with saturable active absorption, or with influx mechanisms, benefit from multiparticulate modified-release dosage form administration. This is because these therapeutic agents have limited periods of time during which they can be absorbed by the intestine, i.e., a limited window of absorption, typically 3-4 hours or less. For example, many di- and tri-peptide mimetic compounds known to be substrates for the PEPT1 intestinal active transport system, exhibit a 3 to 4 hour window of absorption. It has been reported that the antibiotic amoxicillin a substrate for the PEPT1 intestinal active transport system has a limited window of absorption. That limited window of absorption presents a serious challenge to the efficacies of modified release amoxicillin preparations, as is amply illustrated by the reported 1.5 hour Tₘₐₓ for amoxicillin in the commercial product Augmentin XR.

An additional benefit of dosing a multiparticulate modified release dosage form in conformity with the hereinabove-described and hereinbelow-described invention is improved safety. As mentioned above, conventional modified release systems for absorption window limited compounds must front load the majority of release to occur within the limited window of absorption, thereby creating a high Cmax in the plasma profile that could lead to undesirable, toxic side effects, when compared to multiple IR dosing spread over a 24-hour period Compounds such as ciprofloxacin would benefit from the instant invention since the Tₘₐₓ would be extended and the Cmax would be decreased. It is known in the art that a high Cmax of ciprofloxacin leads to serious side effects such a QT prolongation, which can lead to heart failure. By dosing using the multiparticulate modified release dosage form, the AUC would not decrease as it does with other convention modified release systems.

The methods and products described herein will enable the development of once-a-day products utilizing active agents that are not conventionally administered in once-a-day formulations. Such a formulation alternative will likely redound to the benefit of treatment regimens and patient compliance, and will undoubtedly reduce the development costs of a variety of oral pharmaceutical products.

Thus the hereinabove described and hereinbelow described invention addresses the need in the art for modified release formulations of actively transported drugs, or those otherwise exhibiting a window of absorption as stated above, and improved methods of their delivery that extend their Tₘₐₓ, without a significant loss of bioavailability as measured by area under the curve (AUC). And, the hereinabove described and hereinbelow described invention addresses the particular need in the art for modified release formulations of beta-lactam antibiotics and their subclasses, such as penicillins, cephalosporins, and carbapenems, and for improved methods of their delivery, in view of the scarcity of such products that are currently available, and in view of the problems associated with their production, as described in the literature.

Hereinabove-described and hereinbelow-described invention is effective when administered as a single unit dose, such as a tablet or capsule or other dosage form known to one skilled in the art, that disintegrates into multiparticulates in the stomach, or when administered in multiparticulate form such as particulates sprinkled on food (i.e., sprinkled on applesauce), or as beads in a liquid suspension, or when administered in still other forms known to those skilled in the art. Thus the current invention provides added utility not afforded by the prior art for patients that have difficulty swallowing, as frequently experienced by pediatric or geriatric patients.

The instant invention has application to pharmaceutical active agents known to have a limited window of absorption due to various absorption barriers, which could be biological or physico-chemical and, further, could be, but are not limited to poor solubility, low permeability, saturable active absorption or influx mechanisms such as carrier mediated transport, or other barriers known to one skilled in the art that create a small or limited window of absorption. In addition compounds that are converted to their active form via intestinal metabolism, such as some prodrugs, could also be beneficially administered by invention hereinabove-described and hereinbelow-described. The instant invention has application to pharmaceutical active agents known to be substrates of active transporters or subject to a carrier mediated absorption process. The invention has application to all such substrates by increasing the window of absorption. One skilled in the art would appreciate that application insofar as many anti-infectives and other therapeutic agents have been determined to have a limited window of absorption. In a preferred embodiment the pharmaceutical active agent is a substrate for an active transport system. As non-limiting examples of the active transporters for which the pharmaceutical active agents of the present invention may act as substrates there may be mentioned PEPT1, PEPT2, large neutral amino acid transporter, organic cation transporter, monocarboxylic acid transporter, phosphate transporter, and other active transporters known to those of skill in the art. Preferred pharmaceutical active agents for the instant invention are those that are substrates for the PEPT1 and PEPT2 active transport systems. As non-limiting examples of such pharmaceutical active agents there may be mentioned the beta-lactam class of antibiotics, the beta-lactam subclasses penicillins, cephalosporins, and carbapenems and their analogues, valacyclovir, certain ACE inhibitors, and other pharmacologically active agents that are known to those skilled in the art to be substrates for active transport systems.

The invention may also have application to the following, non-limiting anti-infective drug classes: fluoroquinolones and their analogues, aminoglycosides and their analogues, macrolides/ketolides and their analogues, tetracyclines and their analogues, oxazolidinones and their analogues, and sulfonamides and their analogues. The following are further non-limiting examples of antibiotics useful in the present invention: Cefadroxil, cefazolin, cefdinir, cephalexin, cephalothin, cephapirin, cefaclor, cefprozil, cephradine, cefamandole, cefonicid, ceforanide, cefuroxime, cefuroxime axetil, cefixime, cefoperazone, cefotaxime, cefpodoxime, cefpodoxime, proxetil, ceftazidime, ceftibuten, ceftizoxime, ceftriaxone, cefepime, cefinetazole, cefotetan, cefoxitin, loracarbef, imipenem, erythromycin (and erythromycin salts such as estolate, ethylsuccinate, gluceptate, lactobionate, stearate), azithromycin, clarithromycin, dirithromycin, troleandomycin, telithromycin, penicillin V, penicillin salts and complexes, methicillin, nafcillin, oxacillin, cloxacillin, dicloxacillin, amoxicillin, amoxicillin and clavulanate potassium, ampicillin, bacampicillin, carbenicillin indanyl sodium (and other salts of carbenicillin), mezlocillin, piperacillin, piperacillin and tazobactam, ticarcillin, ticarcillin and clavulanate potassium, clindamycin, lincomycin, vancomycin, streptomycin, tobramycin, novobiocin, aminosalicylic acid, capreomycin, cycloserine, ethambutol HCl and other salts, ethibnamide, isoniazid, ciprofloxacin, levofloxacin, lomefloxacin, nalidixic acid, norfloxacin, ofloxacin, sparfloxacin, moxifloxacin, moxifloxacin hydrochloride (and other salts of moxifloxacin), gatifloxacin, gemifloxacin, gemifloxacin mesylate (and other salts of gemifloxacin), sulfacytine, suflamerazine, sulfamethazine, sulfamethizole, sulfasalazine, sulfisoxazole, sulfapyrazine, sulfadiazine, sulfamethoxazole, sulfapyridine, linezolid, tetracycline, doxycycline, okytetracycline, minocycline, demeclocycline, chlortetracycline, metronidazole, methenamine, fosfomycin, nitrofurantoin, trimethoprim, clofazimine, trimoxazole, pentamidine, tigecycline and trimetrexate.

The invention may also have application to the following, non-limiting protease inhibitor class of antivirals and their analogues, the nucleoside reverse trancriptase inhibitor (RTI) class of antivirals and their analogues, the non-nucleoside RTI class of antivirals and their analogues, the nucleotide RTI class of antivirals and their analogues, the viral cellular inhibitor class of antivirals and their analogues, the viral integrase inhibitor class of antivirals and their analogues, the inhibitors of viral/cell fusion and cell entry class of antivirals and their analogues, the DNA-polymerase inhibitor class of antivirals and their analogues, the DNA synthesis inhibitor class of antivirals and their analogues, the immunomodulator class of antivirals and their analogues, the viral nucleic acid release inhibitor class of antivirals and their analogues, the neuraminidase inhibitor class of antivirals and their analogues, the nucleoside analog antiviral class of antivirals and their analogues, the humanized monoclonal antibody class of antivirals and their analogues, neomycin, acyclovir, gancyclovir, cydofovir, amprenavir, fosamprenavir, atazanavir, saquinavir, indinavir, nelfinavir, abacavir, ritonavir, lopinavir, famciclovir, adefovir, emtricitabine, efavirenz, delavirdine, nevirapine, tenofovir, tenofovir disoproxil fumarate (and other salts and esters of tenofovir), oseltamivir, zanamavir, didanosine, foscarnet, zidovudine, lamivudine, stavudine, hydroxyurea, enfuvirtide, T-20, T-1249, PRO-542, SCH-351125, S-1360, interferons, interferon-α2b, interferon-α2a, interferon-alfacon-1, flumantidine, amantidine, ribavirin, ribavirin and interferon-α2b, palivizumab, the azole class of antifungals, the azole subclasses, imidazoles, triazoles, and their analogues, the allylamine class of antifungals and their analogues, the polyene class of antifungals and their analogues, the echinocandin class of antifungals and their analogues, itraconazole, miconazole, clotrimazole, butoconazole, econozole, sulconazole, oxiconazole, tioconazole, bifonazole, croconazole, fenticonazole, isoconazole, omoconazole, terconazole, vibunazole, naftifine, butenafine, nystatin, natamycin, tolnaftate, haloprogin, undecylenic acid, chloroxylenol, ciclopirox, carbolfuchsin, clioquinol, methylrosaniline HCl, selenium sulfide, ketoconazole, fluconazole, itraconazole, voriconazole, posaconazole, caspofungin, anidulofungin, micafungin, terbinafine, amphotericin-b, flucytosine, griseofulvin, epirazolide.

The invention also has application to pharmaceutically active agents that exhibit a pH solubility profile whereby the solubility decreases with increasing pH, or agents that have a window of absorption due to low solubility or a slow dissolution rate. The absorption window of these agents can finally be sufficiently extended so as to allow for the development of effective controlled release systems. Non-limiting examples of such compounds are clarithromycin, ciprofloxacin, ketoconazole, atovaquone, and other BCS class II or IV compounds known to one skilled in the art.

Pharmaceutically active agents that exhibit a limited window of absorption due to permeability limitations such as low permeability or other saturable absorption processes will benefit by the invention. The invention provides a means for these compounds to be developed into effective controlled release delivery systems by delaying achievement of Tmax. Active agents that are highly charged in vivo and hence are mainly absorbed by the paracellular route are particularly suitable for the invention. Non limiting examples of the agents are: quarternary ammonium compounds, neomycin, acyclovir, gancyclovir, itraconazole, epirazolide, doxycycline, and other BCS class III or IV compounds known to one skilled in the art.

Amoxicillin is known to have a 3 to 4 hour window of absorption or less and is further known to be a substrate for the PEPT1 active transport system in the small intestine. Unexpectedly, the instant invention was found to provide an increased AUC for amoxicillin when the modified release formulations was administered in the fed state, as compared to administration of the formulations of amoxicillin in the fasted state. The instant invention doubles the Tₘₐₓ from roughly 3 hours in the fasted state to greater than 6 hours in the fed state, while also increasing the AUC over the fasted state; see the table of pharmacokinetic parameters in example 1 below. Therefore, the instant invention improves upon the bioavailability of amoxicillin, as measured by the increase in AUC. Typically, substantial increases in Tₘₐₓ lead to decreases in bioavailability (a reduction in AUC), making the findings of the current invention all the more unexpected.

The modified release aspects of the modified release dosage form can be accomplished by the use of coatings that are pH dependent or non-pH dependent. As suitable non-pH sensitive delayed release materials there may be mentioned: polyethylene glycol (PEG) with molecular weight above 4,000 daltons (Carbowax, Polyox), waxes such as white wax or bees wax, paraffin, acrylic acid derivatives (Eudragit RL or RS), cellulose acetate, and ethylcellulose. As suitable pH sensitive (enteric) delayed release materials there may be mentioned: cellulose acetate pthalate, Eudragit L or S, and other pthalate salts and acetate succinate salts of cellulose derivatives. The modified release aspects of the modified release dosage form can also be accomplished by the use of sustained release coatings or matrix formulations. As suitable sustained release materials there may be mentioned: ethylcellulose, hydroxypropylmethylcellulose, hydroxypropylcellulose, hydroxyethylcellulose, carboxymethylcellulose, methylcellulose, nitrocellulose, Eudragit RS, and Eudragit RL, Carbopol, or polyethylene glycols with molecular weights in excess of 8,000 daltons.

As hereinabove described and hereinbelow described, embodiments of the invention may comprise dosage forms in addition to the modified release dosage form. Those additional dosage forms may be immediate release dosage forms whereby initiation of release of a pharmaceutically active agent or adjuvant therefrom is not substantially delayed after administration of the pharmaceutical product. As suitable immediate release materials there may be mentioned, microcrystalline cellulose, corn starch, pregelatinized starch, potato starch, rice starch, sodium carboxymethyl starch, hydroxypropylcellulose, hydroxypropylmethylcellulose, hydroxyethylcellulose, ethylcellulose, chitosan, hydroxychitosan, hydroxymethylatedchitosan, cross-linked chitosan, cross-linked hydroxymethyl chitosan, maltodextrin, mannitol, sorbitol, dextrose, maltose, fructose, glucose, levulose, sucrose, polyvinylpyrrolidone (PVP), polyethylene glycols, such a low molecular weight PEGs (PEG2000-8000) and soluble and insoluble inorganic salts such as sodium chloride, calcium sulfate, and organic acids and their salts such as citric acid and sodium citrate.

As hereinabove described and hereinbelow described, embodiments of the invention may comprise dosage forms in addition to the modified release dosage form. Those additional dosage forms may be delayed release dosage forms whereby initiation of release of a pharmaceutically active agent or adjuvant therefrom is substantially delayed after administration of the pharmaceutical product. Such delayed release can be accomplished by the use of coatings that are pH dependent or non-pH dependent. As suitable non-pH sensitive delayed release materials there may be mentioned: polyethylene glycol (PEG) with molecular weight above 4,000 daltons (Carbowax, Polyox), waxes such as white wax or bees wax, paraffin, acrylic acid derivatives (Eudragit RL or RS), cellulose acetate, and ethylcellulose. As suitable pH sensitive (enteric) delayed release materials there may be mentioned: cellulose acetate pthalate, Eudragit L or S, and other pthalate salts of cellulose derivatives.

As hereinabove described and hereinbelow described, embodiments of the invention may comprise dosage forms in addition to the modified release dosage form. Those additional dosage forms may be sustained (extended) release dosage forms whereby initiation of release of a pharmaceutically active agent or adjuvant therefrom may be immediate or may be substantially delayed after administration of the pharmaceutical product, but wherein there is necessarily a release of pharmaceutically active agent or adjuvant therefrom over an extended period of time. As suitable sustained release materials there may be mentioned: ethylcellulose, hydroxypropylmethylcellulose, hydroxypropylcellulose, hydroxyethylcellulose, carboxymethylcellulose, methylcellulose, nitrocellulose, Eudragit RS, and Eudragit RL, Carbopol, or polyethylene glycols with molecular weights in excess of 8,000 daltons.

In addition, it may be useful to have other ingredients in this system to aid in the dissolution of the drug, or the breakdown of the component after ingestion or administration. These ingredients can be surfactants, such as sodium lauryl sulfate, sodium monoglycerate, sorbitan monooleate, sorbitan monooleate, polyoxyethylene sorbitan monooleate, glyceryl monostearate, glyceryl monooleate, glyceryl monobutyrate, one of the non-ionic surfactants such as the Pluronic line of surfactants, or any other material with surface active properties, or any combination of the above.

The invention will be further described with respect to the following example; however, the scope of the invention is not limited thereby.

### EXAMPLE 1

A clinical study compared the administration of 250 mg of two delayed release multiparticulate compositions (Formula B and Formula A) of amoxicillin in the fed and fasted states. This study revealed an unexpected increase in AUC, and hence an increase in bioavailability, in the fed condition, while providing extension of Tₘₐₓ. The current scientific literature reports either a negligible or a negative food effect for amoxicillin, so the increase in absorption was particularly unexpected. The formulations consisted of a pelletized product with a pH dependent coating to provide a delayed release. The manufacturing procedure for each formulation is described below:

Formula B and A pellets are manufactured by first creating a core pellet containing amoxicillin trihydrate. The core pellet is made by creating a wet powder mass by first blending in a suitable planetary or high shear mixer a powder mix consisting of 77% amoxicillin trihydrate powder, 6% avicel PH101, 4% Ac-di-sol, then the wet mass is formed by gradually adding a binder solution consisting of 4% PVP-K30, 3% N-methyl-2-pyrollidone, 6% caprylocaproyl Macrogolglycerides Type 400 and q.s. with water for injection to make a wet mass that contains 20% of the binder solution. The wet mass is then extruded through a 0.6 mm screen. The extrudate is then spheronized until approximately round pellets are obtained. The pellets are then dried in a fluid bed dryer with an inlet temperature of 60° C. until the product temperature reaches 42° C. The dried pellets are then sieved through 20 mesh and 40 mesh screens and the pellets remaining on the 40 mesh screen are collected for futher processing.

Formula B pellets are then created by applying a functional film coating to the core pellets described above. The formula B film coating is manufactured by creating a dispersion consisting of 88.75% water, 6.75% Aqoat HF, 2% talc, 2.3% Triethyl citrate, and 0.2% Sodium Lauryl Sulfate. The dispersion is prepared by adding the TEC and SLS to the water until dissolved. Then, the Aqoat HF is added and mixed for 30 minutes. Next, the talc is added and the dispersion is mixed for one hour before spraying onto the pellets. The dispersion was applied to the pellets in a fluid bed equipped with a wurster column at an inlet air temperature of 70° C., with an atomization air pressure of 2 bar. The disperision is applied at a rate sufficient to maintain a product temperature of approximately 40° C. In the above example 40% weight gain of the coating dispersion is applied to the core pellets. These pellets are then further coated with a topcoat consisting of 2% weight gain of Opadry clear YS-1-7006. Upon completion of the coating, the pellets are sieved and the fraction between 20 and 40 mesh is collected. Finally the pellets are encapsulated into a size 0 hard gelatin capsule for a total dose of 125 mg of amoxicillin.

The formula A pellets are created by applying a different functional film coating to the core pellets described above. The formula A film coating contains 74% water, 12% Eudragit S100, 6% 1N ammonium hydroxide, 6% triethyl citrate and 2% talc. The dispersion is prepared by first adding the Eudragit S100 to the water and mixing a minimum of 5 minutes. Next, the 1N ammonium hydroxide is added to neutralize the polymer, and mixed for 15 minutes. Triethyl citrate is then added and mixed for 30 minutes, and finally talc is added and mixed for 10 minutes. The dispersion is then applied to the core pellets in a wurster column equipped fluid bed at an inlet air termperature of 55° C., and 1.8 bar atomization air. The spray rate is adjusted to maintain a product termperature of 32° C.±5° C. A total of 40% weight gain is applied to the core pellets, which are then sieved to obtain the fraction between 20 and 40 mesh. This fraction is then encapsulate into size 0 hard gelatin capsules for a total dose of 125 mg of amoxicillin.

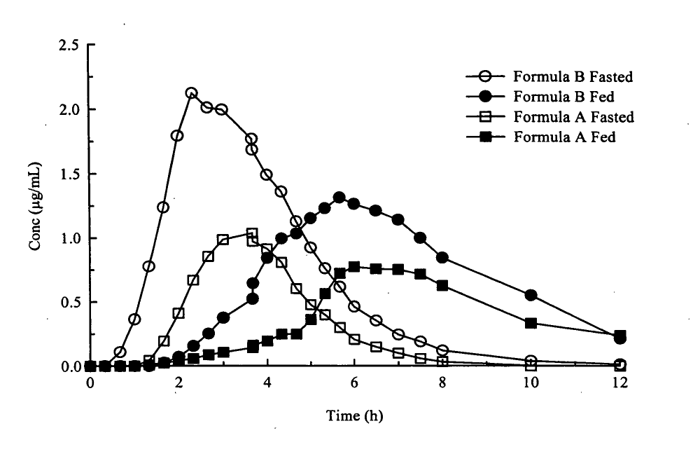

The administration of the formulation with food resulted in an unexpected prolonging of Tₘₐₓ and the more unexpected increase in AUC (bioavailability). The current invention allowed for an extended absorption window as is evidenced by the extension of Tₘₐₓ in the fed state to greater than 6 hours for both formulations (formula B and formula A) as seen in Table I of mean pharmacokinetic parameters and the graph of the mean plasma profiles below, FIG. 1.

TABLE I

| | | AUC₍₀₋ₜ₎ | AUC_{inf} | Cₘₐₓ | Tₘₐₓ |
|---|---|---|---|---|---|
| Treatment | | (μg * hour/mL) | (μg * hour/mL) | (μg/mL) | (hour) |
| B Fasted | Mean | 7.508 | 7.722 | 2.683 | 2.9 |
| B Fed | Mean | 7.539 | 8.269 | 1.716 | 6.2 |
| A Fasted | Mean | 3.319 | 3.515 | 1.418 | 3.2 |
| A Fed | Mean | 4.377 | 5.686 | 1.013 | 6.9 |

Furthermore, upon modeling the above data with Gastro Plus, a pharmacokinetic modeling tool commonly used in the pharmaceutical industry, the absorption time of the fed state was significantly longer than that of the fasted state. The fasted state absorption time that fit the data best was 3.3 hours, or just slightly more than the observed fasted Tmax of both formulations, and the fed absorption time from the model that provided the best fit of the data was 6.5 hours.

1. A method for treating a patient comprising: treating a patient in the fed state with a pharmaceutical product, said pharmaceutical product comprising a modified release dosage form including a pharmaceutically active agent, said pharmaceutically active agent having a limited window of absorption, said modified release dosage form being a dosage form that is released from the stomach into the intestine as particulates, said particulates releasing at least a portion of the pharmaceutically active agent in the small intestine.

2. The method of claim 1, wherein said active agent exhibits a limited window of absorption of 6-8 hours or less.

3. The method of claim 1, wherein said active agent exhibits a limited window of absorption of 2-3 hours or less.

4. The method of claim 1, wherein said pharmaceutical product is in the form of a capsule.

5. The method of claim 1, wherein said pharmaceutical product is in the form of a suspension.

6. The method of claim 1, wherein said pharmaceutical product is in the form of a sprinkle pharmaceutical product.

7. The method of claim 1, wherein said pharmaceutical product is a rapidly disintegrating tablet.

8. The method of claim 1, wherein said pharmaceutical product is a delayed disintegrating tablet.

9. The method of claim 1, wherein said pharmaceutically active agent is amoxicillin.

10. The method of claim 1, wherein said pharmaceutically active agent is ciprofloxacin.

11. The method of claim 1, wherein said pharmaceutically active agent is cephalexin.

12. The method of claim 1, wherein said treating a patient in the fed state with a pharmaceutical product is a once-a-day treating of said patient.

13. A product for treating a patient comprising: (a) a pharmaceutical product comprising a modified release dosage form including a pharmaceutically active agent, said pharmaceutically active agent having a limited window of absorption, said modified release dosage form being a dosage form that is released from the stomach into the intestine as particulates, said particulates releasing at least a portion of the pharmaceutically active agent in the small intestine, and (b) and instructions directing that the pharmaceutical product is to be administered to said patient while said patient is in the fed condition.

14. The product of claim 13, wherein said active agent exhibits a limited window of absorption of 6-8 hours or less.

15. The product of claim 13, wherein said active agent exhibits a limited window of absorption of 2-3 hours or less.

16. The product of claim 13, wherein said pharmaceutical product is in the form of a capsule.

17. The product of claim 13, wherein said pharmaceutical product is in the form of a suspension.

18. The product of claim 13, wherein said pharmaceutical product is in the form of a sprinkle pharmaceutical product.

19. The product of claim 13, wherein said pharmaceutical product is a rapidly disintegrating tablet.

20. The product of claim 13, wherein said pharmaceutical product is a delayed disintegrating tablet.

21. The product of claim 13, wherein said active agent is amoxicillin.

22. The product of claim 13, wherein said active agent is ciprofloxacin.

23. The product of claim 13, wherein said active agent is cephalexin.

24. The product of claim 13, wherein said pharmaceutical product is a once-a-day pharmaceutical product.
